(19) United States
(12) Patent Application Publication
(10) Pub. No.: US 2026/0274870 A1
(43) Pub. Date: Sep. 17, 2026

(54) MATERIAL FOR CHEMICAL VAPOR DEPOSITION, FILM PRODUCTION METHOD, AND FILM

(71) Applicant: L'Air Liquide, Société Anonyme pour l'Etude et l'Etude et l'Exploitation des Procédés Georges Claude, Paris (FR)

(72) Inventors: Sunao KAMIMURA, Yokosuka (JP); Jamie GREER, Cologne (DE); Christian DUSSARRAT, Tokyo (JP)

(21) Appl. No.: 18/871,593

(22) PCT Filed: Jun. 1, 2023
(86) PCT No.: PCT/JP2023/020483

§ 371 Date: Dec. 4, 2024

(30) Foreign Application Priority Data
Jun. 20, 2022 (JP) 2022-098679

(51) Int. Cl. C07F 1/02; C01D 15/04; C23C 16/30

(52) U.S. Cl. CPC C07F 1/02; C01D 15/04; C23C 16/30; C01P 2006/60

(57) ABSTRACT

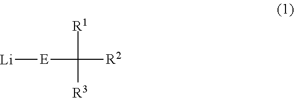

The present invention provides a material for chemical vapor deposition, a film production method, and a film which are novel. Provided is a material for chemical vapor deposition represented by formula (1). (In the formula, E an is an oxygen atom, a sulfur atom, a selenium atom, or a tellurium atom. R¹, R², and R³, are each independently a hydrogen atom or a linear, branched, or cyclic C1-7 organic group, and at least one of R¹, R², and R³ includes a fluorine atom. A case where R¹, R², and R³ are all a hydrogen atom is excluded.

### CROSS REFERENCE TO RELATED APPLICATIONS

This application is a 371 of International Application No. PCT/JP2023/020483, filed Jun. 1, 2023, which claims priority to Japanese Patent Application No. 2022-098679, filed Jun. 20, 2022, the entire contents of which are incorporated herein by reference.

### TECHNICAL FIELD

The present disclosure relates to a chemical vapor deposition material, film production method, and film.

### PRIOR ART

In batteries, durability and high ion conductivity are required. For example, in lithium ion batteries it is necessary to maintain electrode durability and to have a high ion conductivity. With regard to durability, the provision of a film that covers the positive electrode and the negative electrode in lithium ion batteries is known. Lithium phosphate oxynitride (LiPON), lithium niobate (LiNbO₃), lithium titanate (Li₂TiO₃), etc., are examples of compounds contained in the film.

Patent document 1 discloses a chemical vapor deposition material for producing lithium phosphate oxynitride layers.

### PRIOR ART DOCUMENTS

Patent document 1: Japanese unexamined patent 2017-48445.

### SUMMARY OF THE INVENTION

The aim of the present disclosure is the provision of a novel chemical vapor deposition material, film production method, and film.

The present inventors discovered that the abovementioned problem can be solved by a chemical vapor deposition material represented by formula (1) below.

[1] Chemical vapor deposition material represented by formula (1) below.

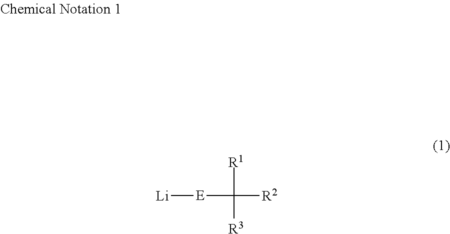

(Here, E is an oxygen atom, sulfur atom, selenium atom or tellurium atom.

R¹, R² and R³ are each independently a hydrogen atom or a C1-7 straight-chain, branched or cyclic organic group,
and at least one of R¹, R² and R³ contains a fluorine atom.

Also, R¹, R² and R³ are not all hydrogen atoms.)

[2] The chemical vapor deposition material described in [1], where R¹, R² and R³ are each independently a hydrogen atom or a C1-5 fluorinated alkyl group, C2-5 alkenyl group, C2-5 fluorinated alkenyl group or C1-5 fluorinated alkoxyalkyl group.

[3] The chemical vapor deposition material described in [1] or [2], where at least one of R¹, R² and R³ is —CF₃, —CF₂CF₃, —CF₂CF₂CF₃, —(CH₂CH═CH₂) or —(CH₂OCH₂CF₃).

[4] The chemical vapor deposition material described in any of [1] to [3], where R¹ is —CF₃.

[5] The chemical vapor deposition material described in any of [1] to [4], where at least one of R² and R³ is a hydrogen atom.

[6] The chemical vapor deposition material described in any of [1] to [5], where E is an oxygen atom.

[7] A film production method provided with a formation step whereby the chemical vapor deposition material described in any of [1] to [6] is vapor-deposited onto a substrate to form a film.

[8] The film production method described [7], where said formation step proceeds in the presence of at least one coreactant chosen from the group consisting of O₂, O₃, H₂O, H₂O₂, NO, NO₂, N₂O, PH3 and TMPO.

[9] The film production method described in [7] or [8], where the temperature of the reactor in said formation step is 150° C. to ≤350° C.

[10] The film production method described in any of [7] to [9], where said film contains a compound represented by LiₓO_{y}F_{z}, wherein

$$2.6 \leq x \leq 4 \text{.3} ,$$

$$1.8 \leq y \leq 2.3 and$$

$$4.3 \leq z \leq 6.6 .$$

[11] The film production method described in any of [7] to [10], where the reflection coefficient of said film is ≥11.1 to ≤1.5.

[12] Film that contains a compound represented by LiₓO_{y}F_{z}, wherein

$$2.6 \leq x \leq 4 \text{.3} ,$$

$$1.8 \leq y \leq 2.3 and$$

$$4.3 \leq z \leq 6.6 .$$

[13] The film described in [12], where the reflection coefficient of said film is ≥1.1 to ≤1.5.

### Advantages of the Invention

The present disclosure allows the provision of a novel chemical vapor deposition material, film production method, and film.

### EMBODIMENTS OF THE INVENTION

Embodiments of the present disclosure are described below, but the present disclosure is not particularly limited to these embodiments, and can be implemented with appropriate modifications. Also, any of the various configurations described below can be implemented in combination.

### Terminology Definitions

Standard abbreviations for elements from the periodic table of elements are used in the present specification. Therefore, elements are represented by these abbreviations. For example, Li denotes lithium, O denotes oxygen, S denotes sulfur, Se denotes selenium, Te denotes tellurium, H denotes hydrogen, C denotes carbon, and F denotes fluorine. The same applies to other elements.

In the present specification, CVD (chemical vapor deposition) denotes the chemical gaseous phase deposition method or the chemical vapor deposition method, and ALD (atomic layer deposition) denotes the atomic layer deposition method. It should be noted that ALD is a type of CVD.

In the present specification, an “organic group” is a group containing at least 1 carbon atom.

In the present specification, a “fluorinated alkyl group” is an alkyl group (functional group) wherein one or more of its hydrogen atoms are substituted by fluorine. Alkyl groups are represented by the general formula CₙH₂ₙ₊₁ (where n is ≥1). “Fluorinated alkyl group” means the same as fluoroalkyl group.

In the present specification, a “fluorinated alkenyl group” is an alkenyl group (functional group) wherein one or more of its hydrogen atoms are substituted by fluorine. Alkenyl groups are represented by the general formula CₙH₂ₙ₋₁ (where n is ≥2).

In the present specification, a “fluorinated alkoxyalkyl group” is an alkoxyalkyl group (functional group) wherein one or more of its hydrogen atoms are substituted by fluorine. Alkoxy groups are represented by the general formula CₙH₂ₙ₊₁O (where n is an integer 1), and alkoxyalkyl groups are represented by the general formula CₙH₂ₙ₊₁O—CₘH₂ₘ (where n and m are each independently an integer 1).

In the present specification, “TMPO” is trimethyl phosphate.

When used in the present specification, the abbreviation “Me” denotes methyl group, the abbreviation “Et” denotes ethyl group; the abbreviation “Pr” denotes propyl group: the abbreviation “ⁿPr” denotes “normal” or straight-chain propyl group; the abbreviation “ⁱPr” denotes isopropyl group; the abbreviation “Bu” denotes butyl group; the abbreviation “ⁿBu” denotes “normal” or straight-chain butyl group; the abbreviation “^{t}Bu” denotes tert-butyl group, also known as 1,1-dimethylethyl; the abbreviation “^{s}Bu” denotes sec-butyl group, also known as 1-methylpropyl; and the abbreviation “ⁱBu” denotes isobutyl group, also known as 2-methylpropyl.

### Chemical Vapor Deposition Material

The chemical vapor deposition material relating to the present embodiments is represented by formula (1) below.

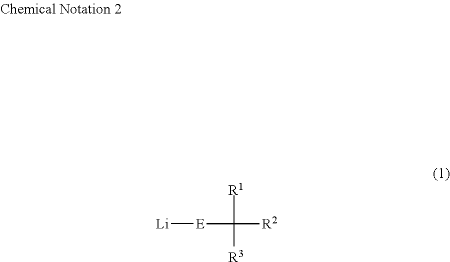

(Here, E is an oxygen atom, sulfur atom, selenium atom or tellurium atom.

R¹, R² and R³ are each independently a hydrogen atom or a C1-7 straight-chain, branched or cyclic organic group,
and at least one of R¹, R² and R³ contains a fluorine atom.

Also, R¹, R² and R³ are not all hydrogen atoms.)

This type of chemical vapor deposition material allows the provision of a novel chemical vapor deposition material.

### Metal

The chemical vapor deposition material has Li (lithium) as its metal element.

### Atom E

Atom E belongs to group 16 of the periodic table. For example, it is an oxygen atom, sulfur atom, selenium atom or tellurium atom. Atom E is preferably an oxygen atom.

Substituent groups R¹, R² and R³

R¹, R² and R³ are each independently a hydrogen atom or a C1-7 straight-chain, branched or cyclic organic group. And at least one of R¹, R² and R³ contains a fluorine atom.

Also, R¹, R² and R³ are not all hydrogen atoms.

Examples of C1-7 straight-chain organic groups are methyl group, ethyl group, propyl group, butyl group, pentyl group, hexyl group, heptyl group, ethenyl group, propenyl group, etc.

Examples of C1-7 branched organic groups are tert-butyl group, sec-butyl group and isobutyl group.

Examples of C1-7 cyclic organic groups are cyclic alkyl groups, alkenyl groups and alkynyl groups.

The abovementioned C1-7 organic groups may have a divalent heteroatom-containing group between a carbon-carbon bond, and some or all of the hydrogen atoms of the abovementioned organic groups may be substituted by monovalent heteroatom-containing groups.

Examples of heteroatoms in the divalent or monovalent heteroatom-containing groups are oxygen atom, nitrogen atom, sulfur atom, phosphorus atom, silicon atom, halogen atom, etc. Examples of halogen atoms are fluorine atom, chlorine atom, bromine atom and iodine atom.

Examples of divalent heteroatom-containing groups are —CO—, —CS—, —NH—, —O—, —S—, —SO₂— and divalent groups containing combinations of these.

Examples of monovalent heteroatom-containing groups are hydroxy group, carboxy group, sulfanyl group, cyano group, nitro group, halogen atoms, etc.

Preferably, R¹, R² and R³ are each independently a hydrogen atom or a C1-5 fluorinated alkyl group, C2-5 alkenyl group, C2-5 fluorinated alkenyl group or C2-5 fluorinated alkoxyalkyl group.

More preferably, at least one of R¹, R² and R³ is —CF₃, —CF₂CF₃, —CF₂CF₂CF₃, —(CH₂CH═CH₂) or —(CH₂OCH₂CF₃).

Even more preferably, R1 is —CF₃.

Particularly preferably, at least one of R² and R³ is a hydrogen atom.

### Specific Examples of Chemical Vapor Deposition Materials

Below are examples of chemical vapor deposition materials.
Li—OCH₂CF₃, Li—OCH₂CF₂CF₃, Li—OC(CH₂CH═C H₂)(CF₃)₂, Li—OC(CF₃)₃, Li—OCH(CF₃)₂, Li—O (CH₂)₂OCH₂CF₃, Li—OC(CH₃)(CF₃)₂, Li—OCH(CH₂OCH₂CF₃)₂

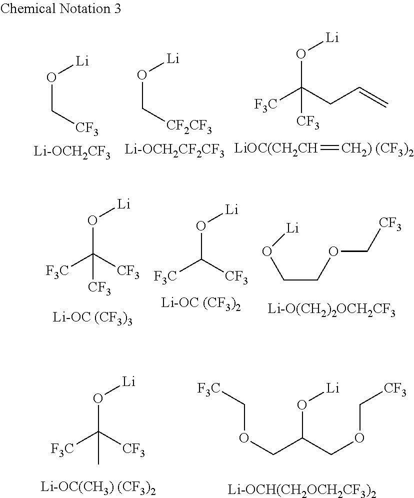

Below are more examples of chemical vapor deposition materials.
LiOC(Et)₂(CF₃), LiOC(Pr)₂(CF₃), LiOC(ⁿBu)₂(CF₃), LiOC(ⁱBu)₂(CF₃), LiOC(^{t}Bu)₂(CF₃), Li OC(^{s}Bu)₂(CF₃), LiOC(MeEt)(CF₃), LiOC(MePr) (CF₃), LiOC(MeⁿEu)(CF₃), LiOC(MeⁱBu)(CF₃), LiOC(Me^{t}Bu)(CF₃), LiOC(Me^{s}Bu)(CF₃), LiOCH₂(CF₂CF₃), LiOCH(CF₂CF₃)₂, LiOC(CH₃)₂(CF₂CF₃), LiOC(MeEt)(CF₂CF₃)LiOC(MePr)(CF₂CF₃), LiOC(MeⁱBu)(CF₂CF₃), LiOC(MenBu)(CF₂CF₃), LiOC(MetBu)(CF₂CF₃), LiOC(Me^{s}Bu)(CF₂CF₃), LiOC(CH₃)(CF₂CF₃)₂, LiOC(CF₂CF₃)₃, LiOC(Et) (CF₃)₂, LiOC(ⁿPr)(CF₃)₂, LiOC(ⁿPr)(CF₃)₂, L iOC(ⁿBu)(CF₃)₂, LiOC(^{t}Bu)(CF₃)₂, LiOC(^{s}Bu) (CF₃)₂, LiOC(ⁱBu)(CF₃)₂, LiO(CH₂)₂OCH₂CF₃, L iO(CH₂)₂OCH(CF₃)₂, LiO(CH₂)₂OC(CF₃)₃, LiO (CH₂)₂OC(CH₃)(CF₃)₂, LiO(CH₂)₂OC(CH₃)₂(CF₃), LiO(CH₂)₂OC(Et)₂(CF₃), LiO(CH₂)₂OC(Pr)₂(CF₃), LiO(CH₂)₂OC(ⁿBu)₂(CF₃), LiO(CH₂)₂OC (ⁱBu)₂(CF₃), LiO(CH₂)₂OC(ⁿBu)₂(CF₃), LiO(C H₂)₂OC(ⁿBu)₂(CF₃), LiO(CH₂)₂OC(MeEt)(CF₃), LiO(CH₂)₂OC(MePr)(F₃), LiO(CH₂)₂OC(MeⁿBu) (CF₃), LiO(CH₂)₂OC(MeiEu)(CF₃), LiO(CH₂)₂O C(MetBu)(CF₃), LiO(CH₂)₂OC(Me^{s}Bu)(CF₃), L iO(CH₂)₂OCH₂(CF₂CF₃), LiO(CH₂)₂OCH(CF2CF₃)₂, LiO(CH₂)₂OC(CH₃)₂(CF₂CF₃), LiO(CH₂)₂OC(M eEt)(CF₂CF₃), LiO(CH₂)₂OC(MePr)(CF₂CF₃), L iO(CH₂)₂OC(MeⁱBu)(CF₂CF₃),
LiO(CH₂)₀ (MeⁿBu)(CF₂CF₃), LiO(CH₂)₂OC(Me^{t}Bu)(CF₂CF₃), LiO(CH₂)₂OC(Me^{s}Bu)(CF₂CF₃), LiO(CH₂)₂OC(CH₃)(CF₂CF₃)₂, LiO(CH₂)₂O C(CF₂CF₃)₃, LiO(CH₂)₂OC(CH₂CH═CH₂)(CF₃)₂, L iO(CH₂)₂OC(Et)(CF₃)₂, LiO(CH₂)₂OC(ⁿPr)(CF₃)₂, LiO(CH₂)₂OC(ⁿPr)(CF₃)₂, LiO(CH₂)₂OC(ⁿB u)(CF₃)₂, LiO(CH₂)₂OC(^{t}Bu)(CF₃)₂, LiO(CH₂)₂OC(^{s}Bu)(CF₃)₂, LiO(CH₂)₂OC(ⁱBu)(CF₃)₂, LiO CH(Me)CH₂OCH₂CF₃, LiOCH(Me)CH₂OCH(CF₃)₂, L iOCH(Me)CH₂OC(CF₃)₃, LiOCH(Me)CH₂OC(CH3) (CF₃)₂, LiOCH(Me)CH₂OC(CH₃)₂(CF₃), LiOCH(M e)CH₂OC(Et)₂(CF₃), LiOCH(Me)CH₂OC(Pr)₂(C F₃), LiOCH(Me)CH₂OC(ⁿBu)₂(CF₃), LiOCH(Me) CH₂OC(ⁱBu)₂(CF₃), LiOCH(Me)CH₂OC(^{t}Bu)₂(CF₃), LiOCH(Me)CH₂OC(^{s}Bu)₂(CF₃), LiOCH(Me)C H₂OC(MeEt)(CF₃), LiOCH(Me)CH₂OC(MePr)(C F₃), LiOCH(Me)CH₂OC(MeⁿBu)(CF₃), LiOCH(Me) CH₂OC(MeⁱBu)(CF₃), LiOCH(Me)CH₂OC(Me^{t}Bu) (CF₃), LiOCH(Me)CH₂OC(Me^{s}Bu)(CF₃), LiOCH (Me)CH₂OCH₂(CF₂CF₃), LiOCH(Me)CH₂OCH(CF₂C F₃)₂, LiOCH(Me)CH₂OC(CH₃)₂(CF₂CF₃), LiOCH (Me)CH₂OC(MeEt)(CF₂CF₃), LiOCH(Me)CH₂OC (MePr)(CF₂CF₃), LiOCH(Me)CH₂OC(MeⁱBu)(CF₂CF₃), LiOCH(Me)CH₂OC(MenBu)(CF₂CF₃), LiOC H(Me)CH₂OC(Me^{t}Bu)(CF₂CF₃), LiOCH(Me)CH₂O C(Me^{s}Bu)(CF₂CF₃) LiOCH(Me)CH₂OC(CH₃)(CF₂CF₃)₂, LiOCH(Me)CH₂OC(CF₂CF₃)₃, LiOCH(Me)C H₂OC(CH₂CH═CH₂)(CF₃)₂,
LiOCH(Me)CH₂OC(Et)(CF₃)₂, LiOCH(Me) CH₂OC(ⁿPr)(CF₃)₂, LiOCH(Me)CH₂OC(ⁱPr)(CF₃)₂, LiOCH(Me)CH₂OC(ⁿBu)(CF₃)₂, LiOCH(Me)CH₂O C(^{t}Bu)(CF₃)₂, LiOCH(Me)CH₂OC(^{s}Bu)(CF₃)₂, L iOCH(Me)CH₂OC(ⁱBu)(CF₃)₂, LiOCH₂CH(Me)OC H₂CF₃, LiOCH₂CH(Me)OCH(CF₃)₂, LiOCH₂CH(Me) OC(CF₃)₃, LiOCH₂CH(Me)OC(CH₃)(CF₃)₂, LiOCH₂CH(Me)OC(CH₃)₂(CF₃), LiOCH₂CH(Me)OC(Et)₂(CF₃), LiOCH₂CH(Me)OC(Pr)₂(CF₃), LiOCH₂CH (Me)OC(ⁿBu)₂(CF₃), LiOCH₂CH(Me)OC(ⁱBu)₂(C F₃), LiOCH₂CH(Me)OC(^{t}Bu)₂(CF₃), LiOCH₂CH(M e)OC(ⁿBu)₂(CF₃), LiOCH₂CH(Me)OC(MeEt)(CF₃), LiOCH₂CH(Me)OC(MePr)(CF₃), LiOCH₂CH(M e)OC(MeⁿBu)(CF₃), LiOCH₂CH(Me)OC(MeⁱBu) (CF₃), LiOCH₂CH(Me)OC(MetBu)(CF₃), LiOCH₂CH(Me)OC(Me^{s}Bu)(CF₃), LiOCH₂CH(Me)OCH₂(C F₂CF₃), LiOCH₂CH(Me)OCH(CF₂CF₃)₂, LiOCH₂CH (Me)OC(CH₃)₂(CF₂CF₃), LiOCH₂CH(Me)OC(MeE t)(CF₂CF₃) LiOCH₂CH(Me)OC(MePr)(CF₂CF₃), LiOCH₂CH(Me)OC(MeⁱBu)(CF₂CF₃), LiOCH₂CH (Me)OC(MeⁿBu)(CF₂CF₃), LiOCH₂CH(Me)OC(Me^{t}Bu)(CF₂CF₃), LiOCH₂CH(Me)OC(Me^{s}Bu)(CF₂CF₃), LiOCH₂CH(Me)OC(CH₃)(CF₂CF₃)₂, LiOCH₂C H(Me)OC(CF₂CF₃)₃, LiOCH₂CH(Me)OC(CH₂CH═C H₂)(CF₃)₂, LiOCH₂CH(Me)OC(Et)(CF₃)₂, LiOCH₂CH(Me)OC(ⁿPr)(CF₃)₂, LiOCH₂CH(Me)OC(ⁿPr) (CF₃)₂, LiOCH₂CH(Me)OC(ⁿBu)(CF₃)₂, LiOCH₂C H(Me)OC(^{t}Bu)(CF₃)₂,
LiOCH₂CH(Me)OC(ⁿBu)(CF₃)₂, LiOCH₂CH (Me)OC(ⁱBu)(CF₃), LiO(CH₂)₃OCH₂CF₃, LiO(C H₂)₃OCH(CF₃)₂, LiO(CH₂)₃OC(CF₃)₃, LiO(CH₂)₃OC(CH₃)(CF₃)₂, LiO(CH₂)₃OC(CH₃)₂(CF₃), LiO (CH₂)₃OC(Et)₂(CF₃), LiO(CH₂)₃OC(Pr)₂(CF₃), LiO(CH₂)₃OC(ⁿBu)₂(CF₃), LiO(CH₂)₃OC(ⁱBu)₂(CF₃), LiO(CH₂)₃OC(_{N}Eu)₂(CF₃), LiO(CH₂)₃OC (^{s}Bu)₂(CF₃), LiO(CH₂)₃OC(MeEt)(CF₃), LiO(C H₂)₃OC(MePr)(CF₃), LiO(CH₂)₃OC(MeⁿBu)(CF₃), LiO(H₂)₃OC(MeⁱBu)(CF₃), LiO(CH₂)₃OC(M e^{t}Bu)(CF₃), LiO(CH₂)₃OC(Me^{s}Bu)(CF₃), LiO(C H₂)₃OCH₂(CF₂CF₃), LiO(CH₂)₃OCH(CF₂CF₃)₂, Li O(CH₂)₃OC(CH₃)₂(CF₂CF₃), LiO(CH₂)₃OC(MeEt) (CF₂CF₃), LiO(CH₂)₃OC(MePr)(CF₂CF₃), LiO(C H₂)₃OC(MeⁱBu)(CF₂CF₃), LiO(CH₂)₃OC(MeⁱBu) (CF₂CF₃), LiO(CH₂)₃OC(MeⁱBu)(CF₂CF₃), LiO (CH₂)₃OC(MeⁱBu)(CF₂CF₃), LiO(CH₂)₃OC(CH₃) (CF₂CF₃)₂, LiO(CH₂)₃OC(CF₂CF₃)₃, LiO(CH₂)₃O C(CH₂CH═CH₂)(CF₃)₂, LiO(CH₂)₃OC(Et)(CF₃)₂, LiO(CH₂)₃OC(ⁿPr)(CF₃)₂, LiO(CH₂)₃OC(ⁱPr) (CF₃)₂, LiO(CH₂)₃OC(ⁿBu)(CF₃)₂, LiO(CH₂)₃OC (^{t}Bu)(CF₃)₂, LiO(CH₂)₃OC(^{s}Bu)(CF₃)₂, LiO(C H₂)₃OC(ⁱBu)(CF₃)₂, LiO(CH₂)₂CH(Me)OCH₂CF₃, LiO(CH₂)₂CH(Me)OCH(CF₃)₂, LiO(CH₂)₂CH(Me) OC(CF₃)₃, LiO(CH₂)₂CH(Me)OC(CH₃)(CF₃)₂, Li O(CH₂)₂CH(Me)OC(CH₃)₂(CF₃), LiO(CH₂)₂CH(M e)OC(Et)₂(CF₃), LiO(CH₂)₂CH(Me)OC(Pr)₂(C F₃), LiO(CH₂)₂CH(Me)OC(ⁿBu)₂(CF₃), LiO(CH₂)₂CH(Me)OC(ⁱBu)₂(CF₃),
LiO(CH₂)₂CH(Me)OC(^{t}Bu)₂(CF₃), LiO(CH₂)₂CH(Me)OC(^{s}Bu)₂(CF₃), LiO(CH₂)₂CH(Me)OC (MeEt)(CF₃), LiO(CH₂)₂CH(Me)OC(MePr)(CF₃), LiO(CH₂)₂CH(Me)OC(MeⁿBu)(CF₃), LiO(CH₂)₂CH(Me)OC(MeⁱBu)(CF₃), LiO(CH₂)₂CH(Me)OC (Me^{t}Bu)(CF₃), LiO(CH₂)₂CH(Me)OC(Me^{s}Bu)(C F₃), LiO(CH₂)₂CH(Me)OCH₂(CF₂CF₃), LiO(CH₂)₂CH(Me)OCH(CF₂CF₃)₂, LiO(CH₂)₂CH(Me)OC(CH₃)₂(CF₂CF₃), LiO(CH₂)₂CH(Me)OC(MeEt)(CF₂C F₃), LiO(CH₂)₂CH(Me)OC(MePr)(CF₂CF₃), LiO (CH₂)₂CH(Me)OC(MeⁿBu)(CF₂CF₃), LiO(CH₂)₂C H(Me)OC(Me^{t}Bu)(CF₂CF₃), LiO(CH₂)₂CH(Me)O C(Me^{t}Bu)(CF₂CF₃), LiO(CH₂)₂CH(Me)OC(MeⁱBu) (CF₂CF₃), LiO(CH₂)2CH(Me)OC(CH₃)(CF₂CF₃)₂, LiO(CH₂)₂CH(Me)OC(CF₂CF₃)₃, LiO(CH₂)₂CH(M e)OC(CH₂CH═CH₂)(CF₃)₂, LiO(CH₂)₂CH(Me)OC (Et)(CF₃)₂, LiO(CH₂)₂CH(Me)OC(ⁿPr)(CF₃)₂, LiO(CH₂)₂CH(Me)OC(iPr)(CF₃)₂, LiO(CH₂)2C H(Me)OC(ⁿBu)(CF₃)₂, LiO(CH₂)₂CH(Me)OC(^{t}Bu) (CF₃)₂, LiO(CH₂)₂CH(Me)OC(ⁿBu)(CF₃)₂, LiO (CH₂)₂CH(Me)OC(ⁱBu)(CF₃)₂, LiOCH(Me)(CH₂)₂OCH₂OCF₃, LiOCH(Me)(OCH₂)₂OCH(CF₃)₂, LiOCH (Me)(CH₂)₂OC(F₃), LiOCH(Me)(CH₂)₂OC(CH₃) (CF₃)₂, LiOCH(Me)(CH₂)₂OC(CH₃)₂(CF₃), LiOC H(Me)(CH₂)₂OC(Et)₂(CF₃) LiOCH(Me)(CH₂)₂O C(Pr)₂(CF₃), LiOCH(Me)(CH₂)₂OC(ⁿBu)₂(CF₃) LiOCH(Me)(CH₂)₂OC(ⁱBu)₂(CF₃), LiOCH(Me) (CH₂)2OC(^{t}Bu)₂(CF₃), LiOCH(Me)(CH₂)₂OC(^{s}B u)₂(CF₃), LiOCH(Me)(CH₂)₂OC(MeEt)(CF₃), L iOCH(Me)(CH₂)₂OC(MePr)(CF₃), LiOCH(Me) (CH₂)₂OC(MeⁿBu)(CF₃) LiOCH(Me)(CH₂)₂OC(M eⁱBu)(CF₃),
LiOCH(Me)(CH₂)₂OC(Me^{t}Bu)(CF₃), LiOC H(Me)(CH₂)₂OC(MeⁱBu)(CF₃), LiOCH(Me)(CH₂)₂OCH₂(CF₂CF₃), LiOCH(Me)(CH₂)₂OCH(CF₂CF₃)₂, LiOCH(Me)(CH₂)₂OC(CH₃)₂(CF₂CF₃), LiOCH(Me) (CH₂)₂OC(MeEt)(CF₂CF₃), LiOCH(Me)(CH₂)₂OC (MePr)(CF₂CF₃), LiOCH(Me)(CH₂)₂OC(MeⁱBu) (CF₂CF₃), LiOCH(Me)(CH₂)₂OC(MenBu)(CF₂CF₃), LiOCH(Me)(CH₂)₂OC(Me^{t}Bu)(CF₂CF₃), LiOC H(Me)(CH₂)₂OC(Me^{s}Bu)(CF₂CF₃), LiOCH(Me) (CH₂)₂OC(CH₃)(CF₂CF₃)₂, LiOCH(Me)(CH₂)₂OC (CF₂CF₃)₃, LiOCH(Me)(CH₂)₂OC(CH₂CH═CH₂)(C F₃)₂, LiOCH(Me)(CH₂)₂OC(Et)(CF₃)₂, LiOCH(M e)(CH₂)₂OC(ⁿPr)(CF₃)₂, LiOCH(Me)(CH₂)₂OC (ⁱPr)(CF₃)₂, LiOCH(Me)(CH₂)₂OC(ⁿBu)(CF₃)₂, LiOCH(Me)(CH₂)₂OC(^{t}Bu)(CF₃)₂, LiOCH(Me) (CH₂)₂OC(ⁿBu)(CF₃)₂, LiOCH(Me)(CH₂)₂OC(ⁱB u)(CF₃)₂, LiOC(Me)₂(CH₂)₂OCH₂CF₃, LiOC(Me)₂(CH₂)₂OCH(CF₃)₂, LiOC(Me)2(CH₂)2OC(CF₃)₃, LiOC(Me)₂(CH₂)₂OC(CH₃)(CF₃)₂, LiOC(Me)₂(C H₂)₂OC(CH₃)₂(CF₃), LiOC(Me)₂(CH₂)₂OC(Et)₂(CF₃), LiOC(Me)₂(CH₂)₂OC(Pr)₂(CF₃), LiOC(M e)₂(CH₂)₂OC(nBU)₂(CF₃), LiOC(Me)₂(CH₂)₂OC (ⁱBu)₂(CF₃), LiOC(Me)₂(CH₂)₂OC(^{t}Bu)(CF₃), LiOC(Me)₂(CH₂)₂OC(^{s}Bu)₂(CF₃),
LiOC(Me)₂(CH₂)₂OC(MeEt)(CF₃), LiOC (Me)₂(CH₂)₂OC(MePr)(CF₃), LiOC(Me)₂(CH₂)₂OC(MeⁿBu)(CF₃), LiOC(Me)₂(CH₂)₂OC(MeiBu) (CF₃), LiOC(Me)₂(CH₂)₂OC(Me^{t}Bu)(CF₃), LiOC (Me)₂(CH₂)₂OC(Me^{e}Bu)(CF₃), LiOC(Me)₂(CH₂)₂OCH₂(CF₂CF₃), LiOC(Me)₂(CH₂)₂OCH(CF₂CF₃)₂, LiOC(Me)₂(CH₂)₂OC(CH3), (CF₂CF₃), LiOC(Me)₂(CH₂)₂OC(MeEt)(CF₂CF₃), LiOC(Me)₂(CH₂)₂O C(MePr)(CF₂CF₃), LiOC(Me)₂(CH₂)₂OC(MeⁱBu) (CF₂CF₃) LiOC(Me)₂(CH₂)₂OC(MeⁿBu)(CF₂CF₃), LiOC(Me)₂(CH₂)₂OC(Me^{t}Eu)(CF₂CF₃), LiOC (Me)₂(CH₂)₂OC(Me^{e}Bu)(CF₂CF₃), LiOC(Me)₂(C H₂)₂OC(CH₃)(CF₂CF₃)₂, LiOC(Me)₂(CH₂)₂OC(C F₂CF₃)₃, LiOC(Me)₂(CH₂)₂OC(CH₂CH═CH₂)(CF₃)₂, LiOC(Me)₂(CH₂)₂OC(Et)(CF₃)₂, LiOC(Me)₂(CH₂)₂OC(ⁿPr)(CF₃)₂, LiOC(Me)₂(CH₂)₂OC(ⁿPr) (CF₃)₂LiOC(Me)₂(CH₂)₂OC(ⁿBu)(CF₃)₂, LiOC (Me)₂(CH₂)₂OC(^{t}Bu)(CF₃)₂, LiOC(Me)₂(CH₂)₂O C(ⁿBu)(CF₃)₂, LiOC(Me)₂(CH₂)₂OC(ⁱBu)(CF₃)₂, LiOC(Me)₂(CH₂)₂OCH₂CF₃, LiOC(Me)₂(CH₂)₂OC H(CF₃)₂, LiOC(Me)₂(CH₂)₂OC(CF₃)₃, LiOC(Me)₂(CH₂)₂OC(CH₃)(CF₃)₂, LiOC(Me)₂(CH₂)₂OC(CH₃)₂(CF₃), LiOC(Me)₂(CH₂)₂OC(CH₂CH═CH₂)(CF₃)₂, LiOC(Me)₂(CH₂)₂OC(Et)(CF₃)₂, LiOC(Me)₂(CH₂)₂OC(ⁿPr)(CF₃)₂, LiOC(Me)₂(CH₂)₂OC(ⁿPr) (CF₃)₂, LiOC(Me)₂(CH₂)₂OC(ⁿBu)(CF₃)₂, LiOC (Me)₂(CH₂)₂OC(^{t}Bu)(CF₃)₂, LiOC(Me)₂(CH₂)₂O C(sBu)(CF₃)₂, LiOC(Me)₂(CH₂)₂OC(ⁱBu)(CF₃)₂, LiOC(CH₂)₂(Me)₂OCH₂CF₃, LiOC(CH₂)₂(Me)₂O CH(CF₃)₂, LiOC(CH₂)₂(Me)₂OC(CF₃)₃, LiOC(CH₂)₂(Me)₂OC(CH₃)(CF₃)₂, LiOC(CH₂)₂(Me)₂OC(C H₃)₂(CF₃), LiOC(CH₂)₂(Me)₂OC(Et)₂(CF₃), Li OC(CH₂)₂(Me)₂OC(Pr)₂(CF₃), LiOC(CH₂)₂(Me)₂OC(ⁿBu)₂(CF₃), LiOC(CH₂)₂(Me)₂OC(ⁱBu)₂(CF₃) LiOC(CH₂)₂(Me)₂OC(^{t}Bu)₂(CF₃) LiOC(CH₂)₂(Me)₂OC(^{e}Bu)₂(CF₃), LiOC(CH₂)₂(Me)₂OC(Me Et)(CF₃) LiOC(CH₂)₂(Me)₂OC(MePr)(CF₃), L iOC(CH₂)₂(Me)₂OC(MeⁿBu)(CF₃), LiOC(CH₂)₂ (Me)₂OC(MeⁱBu)(CF₃),
LiOC(CH₂)₂(Me)₂OC(Me^{t}Bu)(CF₃), LiOC (CH₂)₂(Me)₂OC(Me⁸Bu)(CF₃), LiOC(CH₂)₂(Me)₂OCH₂(CF₂CF₃), LiOC(OH)₂(Me)₂OCH(CF₂CF₃)₂, LiOC(CH₂)₂(Me)₂OC(CH₃)₂(CF₂CF₃), LiOC(CH₂)₂(Me)₂OC(MeEt)(CF₂CF₃), LiOC(CH₂)₂(Me)₂OC (MePr)(CF₂CF₃), LiOC(CH₂)₂(Me)₂OC(MeⁱBu) (CF₂CF₃), LiOC(CH₂)₂(Me)₂OC(MeⁿBu)(CF₂CF₃), LiOC(CH₂)₂(Me)₂OC(MetBu)(CF₂CF₃), LiOC (CH₂)₂(Me)₂OC(Me^{s}Bu)(CF₂CF₃), LiOC(CH₂)₂(Me)₂OC(CH₃)(CF₂F₃)₂LiOC(CH₂)₂(Me)₂OC(C F₂CF₃)₃, LiOC(CH₂)₂(Me)₂OC(CH₂CH═CH₂)(CF₃)₂, LiOC(CH₂)₂(Me)₂OC(Et)(CF₃)₂, LiOC(CH₂)₂(Me)₂OC(ⁿPr)(CF₃)₂, LiOC(CH₂)₂(Me)₂OC(Pr) (CF₃)₂, LiOC(CH₂)₂(Me)₂OC(ⁿBu)(CF₃)₂, LiOC (CH₂)₂(Me)₂OC(^{t}Bu)(CF₃)₂, LiOC(CH₂)₂(Me)₂O C(^{s}Bu)(CF₃)₂, LiOC(CH₂)₂(Me)₂OC(ⁱBu)(CF₃),
LiOCH[(OCH₂)₂OCH₂CF₃]₂, LiOCH[(OCH₂)₂OC H(CF₃)₂]₂, LiOCH[(CH₂)₂OC(CF₃)₃]₂, LiOCH [(OCH₂)₂OC(OCH₃)(CF₃)₂]₂, LiOCH[(OCH₂)₂OC(CH₃)₂(CF₃)]₂, LiOCH[(CH₂)₂OC(Et)₂(F₃)]₂, LiOC H[(CH₂)₂OC(Pr)₂(CF₃)]₂, LiOCH[(CH₂)₂OC(ⁿB u)₂(CF₃)]₂, LiOCH[, (CH₂)₂OC(ⁱBu)₂(CF₃)]₂, Li OCH[(CH₂)₂OC(^{t}Bu)₂(CF₃)]₂, LiOCH[(CH₂)₂OC (^{s}Bu)₂(CF₃)]₂, LiOCH[(CH₂)₂OC(MeEt)(CF₃)]₂, LiOCH[(CH₂)₂OC(MePr)(CF₃)]2, LiOCH[(CH₂)₂OC(MeⁿBu)(CF₃)]₂, LiOCH[(CH₂)₂OC(MeⁱEu) (CF₃)]₂, LiOCH[(CH₂)₂OC(Me^{t}Bu)(CF₃)]₂, LiO CH[(CH₂)₂OC(MeⁿBu)(CF₃)]₂, LiOCH[(CH₂)₂OC H₂(CF₂CF₃)]₂, LiOCH[(CH₂)₂OCH(CF₂CF₃)₂]₂, L iOCH[(CH₂)₂OC(CH₃)₂(CF₂CF₃)]₂LiOCH[(CH₂)₂OC(MeEt)(CF₂CF₃)]₂, LiOCH[(CH₂)₂OC(MePr) (CF₂CF₃)]₂, LiOCH[(CH₂)₂OC(MeⁱBu)(CF₂CF₃)]₂, LiOCH[(CH₂)₂OC(MeⁿBu)(CF₂CF₃)]₂, LiOCH [(CH₂)₂OC(Me^{t}Bu)(CF₂CF₃)]₂, LiOCH[(CH₂)₂O C(Me^{s}Bu)(CF₂CF₃)]₂, LiOCH[(CH₂)₂OC(CH₃)(C F₂CF₃)₂]₂, LiOCH[(CH₂)₂OC(CF₂CF₃)₃]₂, LiOCH [(CH₂)₂OC(CH₂CH═CH₂)(CF₃)₂]₂, LiOCH[(CH₂)₂OC(Et)(CF₃)₂]₂, LiOCH[(CH₂)₂OC(ⁿPr)(CF₃)₂]₂, LiOCH[(CH₂)₂OC(Pr)(CF₃)₂]₂, LiOCH[(CH₂)₂OC(ⁿBu)(CF₃)₂]₂, LiOCH[(CH₂)₂OC(^{t}Bu)(CF₃)₂]₂, LiOCH[(CH₂)₂OC(^{s}Bu)(CF₃)₂]₂, LiOCH[(C H₂)₂OC(ⁱBu)(CF₃)₂]₂, LiOC(Et)₂(CF₂H), LiOC (Et)₂(CFH₂), LiOC(Et)₂(CF₃), LiOCH(CF₂H)₂, L iSCH(CF₃)₂, LiSeCH(CF₃)₂, LiTeCH(CF₃)₂

### Method for Producing Chemical Vapor Deposition Material

Chemical vapor deposition material can usually be produced in accordance with scheme (A) below.

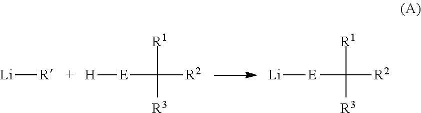

(R′ is a hydrocarbon group. R¹, R² and R³ are as defined for abovementioned formula (1).)

The following procedure is a specific example of the production method. A round-bottomed flask with a dropping funnel attached is prepared. This round-bottomed flask is attached to a distillation unit. An organic solvent having a specified amount of Li source (e.g. n-butyllithium) dissolved therein is introduced into the dropping funnel. Then an organic solvent having a specified amount of carbon source (e.g. HOCH(CF₃)₂) dissolved therein is introduced into the round-bottomed flask. Next, while cooling the dropping funnel to around −30° C. to −10° C., the organic solvent in the dropping funnel is agitated and introduced dropwise into the round-bottomed flask. This affords a precipitate. The organic solvent is removed from the resulting precipitate (e.g. by creating a vacuum in the round-bottomed flask) to obtain a solid. The resulting solid is then subjected to further decrease in pressure (e.g. <0.01 kPa), and the round-bottomed flask is heated to around 100° C. to 180° C. This yields the target chemical vapor deposition material. The various chemical vapor deposition materials of the abovementioned embodiments are obtained by changing the specified carbon source as appropriate.

### Film Production Method

The film production method of the present embodiment includes an introduction step wherein the abovementioned chemical vapor deposition material is introduced into a reactor that houses a substrate; and a formation step wherein at least some of said chemical vapor deposition material is deposited onto said substrate.

### Introduction Step

In this step, the abovementioned chemical vapor deposition material is introduced into a reactor that houses a substrate. The type of substrate onto which a film is to be deposited is selected as appropriate for the end-use. Examples of substrates are Si substrate and electrodes for batteries.

The reactor may be any closed vessel or chamber of a device inside which vapor deposition can be performed. Specific examples include, but are not limited to, parallel plate type reactors, cold wall type reactors, hot wall type reactors, single-wafer reactors, multi-wafer reactors, and other types of deposition system.

Next, gas containing vaporized chemical vapor deposition material is introduced into the reactor. Pure (single) compound or blended (multiple) compounds in the liquid state may be supplied into a vaporizer and vaporized before being introduced into the reactor. Or the chemical vapor deposition material can be vaporized by passing carrier gas through a vessel containing the chemical vapor deposition material, or by bubbling carrier gas through the chemical vapor deposition material. Next, gas containing the carrier gas and vaporized compound(s) is introduced into the reactor. If necessary, the vessel may be heated to a temperature at which the chemical vapor deposition material will have sufficient vapor pressure. There are no limitations on the carrier gas; Ar, He, N₂, and mixtures of these can be used. Or, instead of adopting the method that involves bubbling carrier gas (known as the bubbling method), the compound(s) can be vaporized by direct liquid injection (DLI).

In the introduction step, coreactant can also be introduced into the reactor. The coreactant is preferably chosen from the group consisting of O₂, O₃, H₂O, H₂O₂, NO, N₂O, NO₂, trimethyl phosphate (also referred to as TMPO), oxygen radicals thereof, and mixtures thereof. Other coreactants, such as alcohols, ammonia, polyamines, hydrazine, dimethylethyl phosphoramidates, and sulfates, can also be used.

The vessel is maintained at a temperature in the range of 0° C. to about 150° C., for example. Those skilled in the art will understand that the amount of vaporized compound can be controlled by regulating the vessel temperature by a known method.

The reactor is maintained at a pressure in the range of about 0.5 mTorr to about 20 Torr.

### Formation Step

In the formation step, the abovementioned chemical vapor deposition material is vapor-deposited onto the substrate to form a film.

In this step, in one example of an atomic layer deposition type process, chemical vapor deposition material in the gas phase is introduced into the reactor where it comes into contact with a suitable substrate. Then excess chemical vapor deposition material can be removed from the reactor by purging and/or venting the reactor. Coreactant is introduced into the reactor and reacts in a self-terminating manner with the absorbed chemical vapor deposition material. Excess coreactant is removed from the reactor by purging and/or venting the reactor. When the desired film is a metal oxide film, this 2-stage process may provide the desired film thickness, or this 2-stage process may be repeated until film of the required thickness is obtained.

Or, when the desired film is a metal oxide film, after the abovementioned 2-stage process, the introduction of chemical vapor deposition material vapor into the reactor can be continued. This chemical vapor deposition material is chosen based on the properties of the metal oxide to be deposited. After its introduction into the reactor, the compound comes into contact with the substrate. Excess compound is removed from the reactor by purging and/or venting the reactor. Coreactant may again be introduced into the reactor to react with the chemical vapor deposition material; this coreactant can be the same as or different from the coreactant in the abovementioned 2-stage process. It is preferable to introduce into the reactor a coreactant that is different from the coreactant in the abovementioned 2-stage process. Excess coreactant is removed from the reactor by purging and/or venting the reactor. If the desired film thickness is achieved, this process may be ended. However, if a thicker film is desired, the entire 4-stage process can be repeated. The number of repeat cycles of the 4-stage process is preferably 50-1000 cycles, more preferably 100-800 cycles, and even more preferably 200-600 cycles. Films of the desired composition and thickness can be deposited by alternating the supply of chemical vapor deposition material and coreactant. It is also possible to repeat a 6-stage process using multiple types (e.g. 2 types) of coreactant respectively. The number of repeat cycles of the 6-stage process is, for example, 100 cycles to 600 cycles.

This step may be performed in the presence of at least one coreactant chosen from the group consisting of O₂, O₃, H₂O, H₂O₂, NO, NO₂, N₂O, PH3 and TMPO. Specifically, adding coreactant into the reactor allows a film to form on the substrate. In this step the temperature of the reactor is preferably ≥130° C. to ≤350° C., more preferably ≥130° C. to ≤300° C., even more preferably ≥130° C. to ≤250° C., and particularly preferably ≥130° C. to ≤200° C. By using a relatively low reactor temperature it is possible to form films on heat-sensitive substrates. For example, it is possible to form films on lithium ion battery electrodes.

In another embodiment, the temperature of the reactor in this step is preferably ≥130° C. to ≤350° C., more preferably ≥150° C. to ≤350° C., even more preferably ≥170° C. to ≤350° C., and particularly preferably ≥200° C. to ≤350° C. By using a relatively high reactor temperature it is possible to form films at high growth rates.

In this embodiment the film growth rate per cycle is preferably ≥0.05 Å to ≤0.7 Å, more preferably ≥0.08 Å to ≤0.6 Å, and even more preferably 0.1 Å to ≤0.5 Å. The faster the film growth rate, the more efficiently film can be formed.

### Compounds Contained in the Film

Film produced by the film production method described above contains metal oxyfluoride. Here, the metal oxyfluoride contains Li, oxygen and fluorine.

Film produced by the film production method described above contains a compound represented by LiₓO_{y}F_{z}. Here, the compound is such that 2.6≤x≤4.3, 1.8≤y≤2.3, and 4.3≤z≤6.6. The composition of the compound can be calculated from analysis results obtained using a commercially available elemental analyzer, and stoichiometric calculation using those analysis results. It should be noted that the compound contained in the film may be substantially LiₓO_{y}F_{z}(≥99% by mass LiₓO_{y}F_{z}) or may contain impurities (e.g. LiOF and/or LiCF).

The film produced by the film production method described above has a reflection coefficient of ≥1.1 to ≤1.5. The reflection coefficient can be measured using a commercially available spectroscopic ellipsometer (i.e., an ellipsometer).

### Film

The film in the present embodiment contains a compound represented by LiₓO_{y}F_{z}, wherein 2.6≤x≤4.3, 1.8≤y≤3, and 4.3≤z≤6.6. The composition of the compound can be calculated from analysis results obtained using a commercially available elemental analyzer, and stoichiometric calculation using those analysis results. Here, the compound LiₓO_{y}F_{z} contained in the film constitutes preferably ≥50% by mass, more preferably ≥60% by mass, even more preferably ≥70% by mass, and particularly preferably ≥90% by mass, thereof.

The film of the present embodiment has a reflection coefficient of ≥1.1 to ≤1.5. The reflection coefficient can be measured using a commercially available spectroscopic ellipsometer. Because it has a relatively low reflection coefficient, the film will have multiple uses.

The film thickness is preferably ≥0.1 nm to ≤100 nm, more preferably ≥1 nm to ≤50 nm.

### Uses

The chemical vapor deposition material and film of the present embodiment are useful in the production of lithium oxide, lithium oxyfluoride or alkali-containing mixed metal oxyfluoride films, for various uses such as coating lithium metal, and coating cathodes and anodes in lithium batteries. Lithium oxyfluoride films have improved resistance to oxidizing agents; this improved resistance can bestow the solid electrolyte mesophase with improved resistance to the gradual deterioration that decreases battery-charging efficiency.

### EXAMPLES

The examples below are provided to illustrate use the disclosure of the present specification, and it should be fully understood that any specific embodiment or group of embodiments of the present invention may not necessarily afford all of the advantages of the process described in the present specification. Although specific embodiments and examples are disclosed below, it will be understood by those skilled in the art that the present invention includes obvious modifications and exceeds the specifically disclosed inventive embodiments and/or uses. Therefore, it should be understood that the scope of the disclosed present invention is not limited to the specific embodiments described below.

### Synthesis of Chemical Vapor Deposition Material

A 500 mL round-bottomed flask with an attached 100 mL dropping funnel was prepared. A distillation unit was attached to this round-bottomed flask. A 250 mL round-bottomed flask was attached to the distillation unit. Then 58 mL n-hexane (n-hexane manufactured by Kanto Chemical Co., Inc.) having 2.6 M n-butyllithium (manufactured by Kanto Chemical Co., Inc.) dissolved therein was introduced into the dropping funnel. Then 200 mL n-hexane having 150 mmol HOCH(CF₃)₂ (manufactured by Tokyo Chemical Industry Co., Ltd.) dissolved therein was introduced into the 500 mL round-bottomed flask. Next, while cooling the dropping funnel to −20° C., the n-hexane in the dropping funnel was agitated and introduced dropwise into the 500 mL round-bottomed flask. This afforded a precipitate. The n-hexane was removed from the resulting precipitate, to obtain a solid. The resulting solid was then subjected to further decrease in pressure to vacuum (<0.01 kPa), and the 500 mL round-bottomed flask was heated to around 150° C. This yielded the target chemical vapor deposition material.

The ¹H NMR (C6D6, 400 MHz) results were as follows. From these results it was confirmed that the target chemical vapor deposition material was Li—O—CH(CF₃)₂. A Bruker Ascend 400 MHz NMR unit was used for the NMR measurements.

1H NMR (C6D6, 40 MHz): δ 3.988 (1H, d, O—C H(CF₃)₂); 13C NMR (C6D6/D6-DMSO 101 MHz): 68.827 (m, O—CH(CF₃)₂), 123.1 (q, 1 JCF=288. 6 Hz, O—CH(CF₃)₂); 19F NMR (C6D6, 376 MHz): −78.415 (s, Li—O—CH(CF₃)₂); 1Li NMR (C6D6, 1 56 MHz): 0.211 (s, Li—O—CH(CF₃)₂).

### Film Formation

### Sample 1

The Li—O—CH(CF₃)₂ synthesized in “Synthesis of chemical vapor deposition material” described above was introduced into a vessel (purchased from Japan Advanced Chemicals Co., Ltd). Then the inside of the vessel was set at 78° C. At this point, the pressure was 10 Torr. The vessel was connected to allow the gas in the vessel to be supplied to the reactor.

Next, Si substrate (produced by Mitsubishi Materials Corporation, product name: P-type Silicon Wafer) that had been washed using 1 vol % HF solution was introduced into the reactor. The temperature and pressure inside the reactor were set at 200° C. and 1 Torr, the atmosphere surrounding the substrate was changed as described below, and 400 cycles were repeated. This resulted in the formation of a film.

### Changes of Atmosphere

4 sccm LiO—CH(CF₃)₂ (2.5 s)

300 sccm nitrogen gas (30 s)

20 sccm H₂O gas (1 s)

300 sccm nitrogen gas (30 s)

This series (1) to (4) constituted 1 cycle.

### Sample 2

A film was formed by the same method as for Sample 1, except that in “Changes of atmosphere”, step (1) was 5 s.

### Sample 3

A film was formed by the same method as for Sample 1, except that in “Changes of atmosphere”, step (1) was 10 s.

### Sample 4

A film was formed by the same method as for Sample 1, except that in “Changes of atmosphere”, step (1) was 15 s.

### Sample 5

A film was formed by the same method as for Sample 1, except that the temperature inside the reactor was 175° C.

### Sample 6

A film was formed by the same method as for Sample 1, except for the following.
The temperature inside the reactor was set at 175° C.
In “Changes of atmosphere”, (1) was 5 s

### Sample 7

A film was formed by the same method as for Sample 1, except for the following.
The temperature inside the reactor was set at 175° C.
In “Changes of atmosphere”, (1) was 10 s

### Sample 8

A film was formed by the same method as for Sample 1, except for the following.
The temperature inside the reactor was set at 175° C.
In “Changes of atmosphere”, (1) was 15 s

### Sample 9

A film was formed by the same method as for Sample 1, except for the following.
The temperature inside the reactor was set at 150° C.
In “Changes of atmosphere”, (1) was 15 s
The number of cycles was 600

### Sample 10

The Li—O—CH(CF₃)₂ synthesized in “Synthesis of chemical vapor deposition material” described above was introduced into a first vessel (purchased from Japan Advanced Chemicals Co., Ltd). Then the inside of the first vessel was set at 78° C. At this point, the pressure was 20 Torr. TMPO (manufactured by Aldrich) was added into a second vessel (purchased from Japan Advanced Chemicals Co., Ltd). Then the inside of the second vessel was set at 80° C. At this point, the pressure was 20 Torr. The first vessel and the second vessel were connected to allow gas to be supplied to the reactor.

Next, Si substrate (produced by Mitsubishi Materials Corporation, product name: P-type Silicon Wafer) that had been washed using 1 vol % HF solution was introduced into the reactor. The temperature and pressure inside the reactor were set at 200° C. and 1 Torr, the atmosphere surrounding the substrate was changed as described below, and 400 cycles were repeated. This resulted in the formation of a film.

### Changes of Atmosphere

(1) 1 sccm Li—O—CH(CF₃)₂ (5 s)
(2) 300 sccm nitrogen gas (5 s)
(3) 30 sccm TMPO (5 s)
(4) 300 sccm nitrogen gas (5 s)
(5) 50 sccm ozone gas (5 s)
(6) 300 sccm nitrogen gas (5 s)

This series (1) to (6) constituted 1 cycle.

### Sample 11

A film was formed by the same method as for Sample 10, except that in “Changes of atmosphere”, step (1) was 20 s.

### Sample 12

A film was formed by the same method as for Sample 10, except that the temperature inside the reactor was 250° C.

### Sample 13

A film was formed by the same method as for Sample 10, except for the following.
The temperature inside the reactor was set at 250° C.
In “Changes of atmosphere”, step (1) was 10 s

### Sample 14

A film was formed by the same method as for Sample 10, except for the following.
The temperature inside the reactor was set at 250° C.
In “Changes of atmosphere”, step (1) was 15 s

### Sample 15

A film was formed by the same method as for Sample 10, except for the following.
The temperature inside the reactor was set at 250° C.
In “Changes of atmosphere”, step (1) was 20 s

### Sample 16

A film was formed by the same method as for Sample 10, except for the following.
The temperature inside the reactor was set at 250° C.
In “Changes of atmosphere”, step (1) was 25 s

### Sample 17

A film was formed by the same method as for Sample 10, except that the temperature inside the reactor was 275° C.

### Sample 18

A film was formed by the same method as for Sample 10, except for the following.
The temperature inside the reactor was set at 275° C.
In “Changes of atmosphere”, step (1) was 10 s

### Sample 19

A film was formed by the same method as for Sample 10, except for the following.
The temperature inside the reactor was set at 275° C.
In “Changes of atmosphere”, step (1) was 15 s

### Sample 20

A film was formed by the same method as for Sample 10, except for the following.
The temperature inside the reactor was set at 275° C.
In “Changes of atmosphere”, step (1) was 20 s

### Sample 21

A film was formed by the same method as for Sample 10, except for the following.
The temperature inside the reactor was set at 275° C.
In “Changes of atmosphere”, step (1) was 25 s

### Sample 22

A film was formed by the same method as for Sample 10, except that the temperature inside the reactor was 300° C.

### Sample 23

A film was formed by the same method as for Sample 10, except for the following.
The temperature inside the reactor was set at 300° C.
In “Changes of atmosphere”, step (1) was 10 s

### Sample 24

A film was formed by the same method as for Sample 10, except for the following.
The temperature inside the reactor was set at 300° C.
In “Changes of atmosphere”, step (1) was 15 s

### Sample 25

A film was formed by the same method as for Sample 10, except for the following.
The temperature inside the reactor was set at 300° C.
In “Changes of atmosphere”, step (1) was 20 s

### Sample 26

The Li—O—CH(CF₃)₂ synthesized in “Synthesis of chemical vapor deposition material” described above was introduced into a vessel (purchased from Japan Advanced Chemicals Co., Ltd). Then the inside of the vessel was set at 78° C. At this point, the pressure was 20 Torr. The vessel was connected to allow gas to be supplied to the reactor.

Next, Si substrate (produced by Mitsubishi Materials Corporation, product name: P-type Silicon Wafer) that had been washed using 1 vol % HF solution was introduced into the reactor. The temperature and pressure inside the reactor were set at 200° C. and 1 Torr, the atmosphere surrounding the substrate was changed as described below, and 400 cycles were repeated. This resulted in the formation of a film.

### Changes of Atmosphere

(1) 1 sccm Li—O—CH(CF₃)₂ (5 s)
(2) 300 sccm nitrogen gas (5 s)
(3) 50 sccm ozone gas (5 s)
(4) 300 sccm nitrogen gas (5 s)

This series (1) to (4) constituted 1 cycle.

### Sample 27

A film was formed by the same method as for Sample 26, except that in “Changes of atmosphere”, step (1) was 20 s.

### Sample 28

A film was formed by the same method as for Sample 26, except that the temperature inside the reactor was 250° C.

### Sample 29

A film was formed by the same method as for Sample 26, except for the following.
The temperature inside the reactor was set at 250° C.
In “Changes of atmosphere”, (1) was 20 s

### Sample 30

A film was formed by the same method as for Sample 26, except that the temperature inside the reactor was 275° C.

### Sample 31

A film was formed by the same method as for Sample 26, except for the following.
The temperature inside the reactor was set at 275° C.
In “Changes of atmosphere”, step (1) was 20 s

### Sample 32

A film was formed by the same method as for Sample 26, except that the temperature inside the reactor was 300° C.

### Sample 33

A film was formed by the same method as for Sample 26, except for the following.
The temperature inside the reactor was set at 300° C.
In “Changes of atmosphere”, step (1) was 20 s

### Analysis of the Film

The films of Samples 1 to 33 were analyzed using an ellipsometer (manufactured by Semilab, product name: SE-2000). The analysis results (film growth rate (A (Angstrom)/cycle) and reflection coefficient) for each sample are compiled in Table 1 to Table 3. In the tables, “-” denotes that that component was not used. Also, in the tables, Li-HFIP denotes Li—O—CH(CF₃)₂.

TABLE 1

| | Sample | 1 | 2 | 3 | 4 | 5 | 6 | 7 | 8 | 9 |
|---|---|---|---|---|---|---|---|---|---|---|
| Test conditions | Reactor | 200 | 200 | 200 | 200 | 175 | 175 | 175 | 175 | 150 |
| | temperature (° C.) | | | | | | | | | |
| Type of coreactant | | H₂O | | | | | | | | |
| Chemical vapor deposition material- | Li-HFIP (4 sccm) | 2.5 | 5 | 10 | 15 | 2.5 | 5 | 10 | 15 | 15 |
| containing gas flow time (s) | Li-HFIP (1 sccm) | — | — | — | — | — | — | — | — | — |
| Analysis results | Growth rate | 0.28 | 0.3 | 0.31 | 0.32 | 0.159 | 0.178 | 0.174 | 0.177 | 0.118 |
| | (Angstrom/cycle) | | | | | | | | | |
| | Reflection | 1.41 | 1.42 | 1.42 | 1.42 | 1.4 | 1.42 | 1.42 | 1.42 | 1.43 |
| | coefficient | | | | | | | | | |

TABLE 2

| | Sample | 10 | 11 | 12 | 13 | 14 | 15 | 16 | 17 |
|---|---|---|---|---|---|---|---|---|---|
| Test | Reactor | 200 | 200 | 250 | 250 | 250 | 250 | 250 | 275 |
| conditions | temperature (° C.) | | | | | | | | |
| Type of | | TMPO + O₃ | | | | | | | |
| coreactant | | | | | | | | | |
| Chemical | Li-HFIP (4 sccm) | — | — | — | — | — | — | — | — |
| vapor | Li-HFIP (1 sccm) | 5 | 20 | 5 | 10 | 15 | 20 | 25 | 5 |
| deposition | | | | | | | | | |
| material- | | | | | | | | | |
| containing | | | | | | | | | |
| gas flow | | | | | | | | | |
| time (s) | | | | | | | | | |
| Analysis | Growth rate | 0.12 | 0.09 | 0.22 | 0.28 | 0.46 | 0.42 | 0.4 | 0.36 |
| results | (Angstrom/cycle) | | | | | | | | |
| | Reflection | 1.37 | 1.28 | 1.33 | 1.36 | 1.33 | 1.33 | 1.36 | 1.33 |
| | coefficient | | | | | | | | |
| | Sample | 18 | 19 | 20 | 21 | 22 | 23 | 24 | 25 |
| Test | Reactor | 275 | 275 | 275 | 275 | 300 | 300 | 300 | 300 |
| conditions | temperature (° C.) | | | | | | | | |
| Type of | | TMPO + O₃ | | | | | | | |
| coreactant | | | | | | | | | |
| Chemical | Li-HFIP (4 sccm) | — | — | — | — | — | — | — | — |
| vapor | Li-HFIP (1 sccm) | 10 | 15 | 20 | 25 | 5 | 10 | 15 | 20 |
| deposition | | | | | | | | | |
| material- | | | | | | | | | |
| containing | | | | | | | | | |
| gas flow | | | | | | | | | |
| time (s) | | | | | | | | | |
| Analysis | Growth rate | 0.46 | 0.49 | 0.51 | 0.51 | 0.61 | 0.57 | 0.46 | 0.44 |
| results | (Angstrom/cycle) | | | | | | | | |
| | Reflection | 1.33 | 1.33 | 1.36 | 1.36 | 1.39 | 1.39 | 1.39 | 1.39 |
| | coefficient | | | | | | | | |

TABLE 3

| | Sample | 26 | 27 | 28 | 29 | 30 | 31 | 32 | 33 |
|---|---|---|---|---|---|---|---|---|---|
| Test conditions | Reactor | 200 | 200 | 250 | 250 | 275 | 275 | 300 | 300 |
| | temperature (° C.) | | | | | | | | |
| Type of coreactant | | O₃ | | | | | | | |
| Chemical vapor deposition material- | Li-HFIP (4 sccm) | — | — | — | — | — | — | — | — |
| containing gas flow time (s) | Li-HFIP (1 sccm) | 5 | 20 | 5 | 20 | 5 | 20 | 5 | 20 |
| Analysis results | Growth rate | 0.11 | 0.07 | 0.24 | 0.18 | 0.27 | 0.19 | 0.28 | 0.12 |
| | (Angstrom/cycle) | | | | | | | | |
| | Reflection | 1.35 | 1.21 | 1.23 | 1.36 | 1.32 | 1.31 | 1.32 | 1.26 |
| | coefficient | | | | | | | | |

1. A chemical vapor deposition material represented by formula (1) below 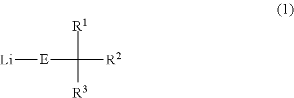
wherein E is an oxygen atom, sulfur atom, selenium atom or tellurium atom; R¹, R² and R³ are each independently a hydrogen atom or a C1-7 straight-chain, branched or cyclic organic group; at least one of R¹, R² and R³ contains a fluorine atom; and R¹, R² and R³ are not all hydrogen atoms.

2. The chemical vapor deposition material claimed in claim 1, where R¹, R² and R³ are each independently a hydrogen atom or a C1-5 fluorinated alkyl group, C2-5 alkenyl group, C2-5 fluorinated alkenyl group or C2-5 fluorinated alkoxyalkyl group.

3. The chemical vapor deposition material claimed in claim 2, where at least one of R¹, R² and R³ is —CF₃, —CF₂CF₃, —CF₂CF₂CF₃, —(CH₂CH═CH₂) or —(CH₂OCH₂CF₃).

4. The chemical vapor deposition material claimed in claim 3, where R1 is —CF₃.

5. The chemical vapor deposition material claimed in claim 4, where at least one of R² and R³ is a hydrogen atom.

6. The chemical vapor deposition material claimed in claim 1, where E is an oxygen atom.

7. A film production method provided with a formation step whereby the chemical vapor deposition material of claim 1 is vapor-deposited onto a substrate to form a film.

8. The film production method claimed in claim 7, where said formation step proceeds in the presence of at least one coreactant chosen from the group consisting of O₂, O₃, H₂O, H₂O₂, NO, NO₂, N₂O, PH₃ and (CH₃)₃PO₄ (TMPO).

9. The film production method claimed in claim 8, where a temperature of a reactor in said formation step is ≥130° C. to ≤350° C.

10. The film production method claimed in claim 7, where said film contains a compound represented by LiₓO_{y}F_{z}, wherein
$$2.6 \leq x \leq 4 \text{.3} ,$$
$$1.8 \leq y \leq 2.3 , and$$
$$4.3 \leq z \leq 6.6 .$$

11. The film production method claimed in claim 10, where the refractive index of said film is ≥1.1 to ≤1.5.

12. A film that contains a compound represented by LiₓO_{y}F_{z}, wherein
$$2.6 \leq x \leq 4 \text{.3} ,$$
$$1.8 \leq y \leq 2.3 , and$$
$$4.3 \leq z \leq 6.6 .$$

13. The film claimed in claim 12, where the refractive index of said film is ≥1.1 to ≤1.5.
